(19) United States
(12) Patent Application Publication
(10) Pub. No.: US 2014/0180083 A1
(43) Pub. Date: Jun. 26, 2014

(54) SYSTEM AND METHOD FOR FLUSH-TRIGGERED IMAGING

(71) Applicant: VOLCANO CORPORATION, San Diego, CA (US)

(72) Inventor: Paul Hoseit, El Dorado Hills, CA (US)

(73) Assignee: VOLCANO CORPORATION, San Diego, CA (US)

(21) Appl. No.: 14/109,019

(22) Filed: Dec. 17, 2013

Related U.S. Application Data
(60) Provisional application No. 61/745,299, filed on Dec. 21, 2012.

(51) Int. Cl. A61B 5/00; A61B 8/00

(52) U.S. Cl. CPC A61B 5/7285; A61B 5/0073; A61B 8/543
USPC 600/428; 600/431

(57) ABSTRACT

The invention generally relates to intravascular imaging and methods of improved image quality by triggering image operations with a vessel flush. The invention provides systems and methods for intravascular imaging in which a flush such as the influx of clear saline or radiopaque dye triggers the imaging operation. The flush is detected by a mechanism—such as a pressure sensor or optical device on the imaging catheter, an external angiography system, or other device—that uses detection as a trigger to initiate imaging. Thus, when the blood is flushed, the catheter automatically takes a picture of the vessel wall.

### CROSS-REFERENCE TO RELATED APPLICATION

This application claims priority to, and the benefit of, U.S. Provisional Patent Application Ser. No. 61/745,299, filed Dec. 21, 2012, the contents of which are incorporated by reference.

### FIELD OF THE INVENTION

The invention generally relates to intravascular imaging and methods of improved image quality by triggering image operations with a detection of a blood vessel flush.

### BACKGROUND

Intravascular imaging refers to medical procedure that involve inserting a catheter into a patient's blood vessels to examine those vessels from within. Intravascular imaging procedures are critical for the detection and treatment of life threatening plaque. In many cases, vulnerable arterial plaques are otherwise asymptomatic until they break up and a clot flows deep into a patient's vessels or even into the brain, causing a heart attack or stroke. Existing imaging technologies that can be used to detect such plaques include intravascular ultrasound (IVUS) and optical coherence tomography (OCT). However, for each of these technologies, the presence of blood in the vessels interferes with getting a clean image.

A good quality image can be obtained by temporarily replacing the blood with a clear saline solution. In OCT, this is a regular part of the procedure, and flushing the blood with saline shows potential for improving image quality in high-frequency IVUS, as well. Unfortunately, coordinating the timing between flushing the vessel and taking the picture is proving difficult. As it stands, one person in the operating room injects the saline while another person triggers the imaging operation. Through experience, and by trying to time their efforts just-so, it is hoped that the IVUS or OCT catheter will travel through its image capture “pullback” just as the clear saline flow through the same region of vessel. However, the results can be poor. If the timing is off by a little bit, the image will be taken from within the relatively opaque blood. This can produce images that do not clearly reveals arterial plaque and require do-overs.

### SUMMARY

The invention provides systems and methods for intravascular imaging in which the influx of a flush solution (e.g., of saline or a radiopaque dye) automatically triggers an imaging operation. The displacement of blood by saline or dye can be detected by a mechanism such as a pressure sensor or optical device on the imaging catheter, an external angiography system, or other device. The detection mechanism is operably coupled to the imaging system so that the imaging system can use the detection event as the direct trigger to initiate imaging. Thus, when the saline flows in, the IVUS or OCT pullback begins, and the imaging tip of the catheter takes a picture of the vessel wall through the clear saline. This has particular application in OCT, which gets the best signal through saline, and high-frequency IVUS, in which blood produces a speckle noise that interferes with lumen border detection. Since the imaging operation is performed automatically while the saline has flushed the blood from the vessel, those functions do not need to be manually coordinated by different people trying to time their work together outside of the patient. Since manual timing is not required, there will be few instances of inadvertent imaging from within blood, and therefore better, more useful images will be consistently produced with few do-overs. Due to the consistent, high-quality images produced using systems and methods of the invention, arterial plaque can be identified and treated in more patients in good time prior to adverse coronary events.

In certain aspects, the invention provides a method of intravascular imaging that includes positioning a distal end of an imaging catheter within a blood vessel (e.g., immersed in blood), providing an influx of a solution to flush the blood from the vessel, detecting the influx, and using the detection of the influx of the solution to trigger an imaging operation by means of an imaging system connected to a proximal end of the catheter. The influx can be detected from within the vessel using, for example, the optics of an OCT system or an pressure sensor on an IVUS system. The influx may be detected from outside of the patient's body using, for example, an angiogram. When the influx is detected, the imaging operation that is initiated can include starting a pullback of the catheter, a rotation, or both. In some embodiments, the imaging system is configured to initiate a catheter pullback automatically and in direct response to the detection of the influx.

In related aspects, the invention provides a system for intravascular imaging that include an imaging instrument; a catheter connected to the imaging instrument and configured for insertion into a blood vessel; a lumen extending along the catheter for delivery of a solution to flush blood from the blood vessel; and a sensor to detect an influx of the solution. The system is configured to use the detection of the influx of the solution to trigger an imaging operation with the imaging catheter.

### BRIEF DESCRIPTION OF THE DRAWINGS

FIG. 1 shows an intravascular imaging system.

FIG. 2 is a diagram of components of an OCT system.

FIG. 3 gives a detailed view of an OCT imaging engine.

FIG. 4 is a schematic of an OCT patient interface module.

FIG. 5 shows a pattern that an OCT imaging fiber traces during a pullback.

FIG. 6 diagrams a pattern of scan lines produced by an imaging operation.

FIG. 7 is a reproduction of a display from an intravascular imaging system.

FIG. 8 is an illustration of a display from an intravascular imaging system.

FIG. 9 diagrams a method of flush-triggered imaging according to certain embodiments.

FIG. 10 shows a method for angio-triggered imaging.

FIG. 11 gives a diagram of components of an IVUS system.

FIG. 12 shows an IVUS control station.

FIG. 13 is a schematic of components within an IVUS system.

FIG. 14 is a diagram of a flush-triggered IVUS imaging method.

FIG. 15 illustrates angio-triggered IVUS imaging.

### DETAILED DESCRIPTION

The invention provides systems and methods for coordinating operations during intravascular imaging. Any intravascular imaging system may be used in systems and methods of the invention. Systems and methods of the invention have application in intravascular imaging methodologies such as intravascular ultrasound (IVUS) and optical coherence tomography (OCT) among others that produce a three-dimensional image of a vessel.

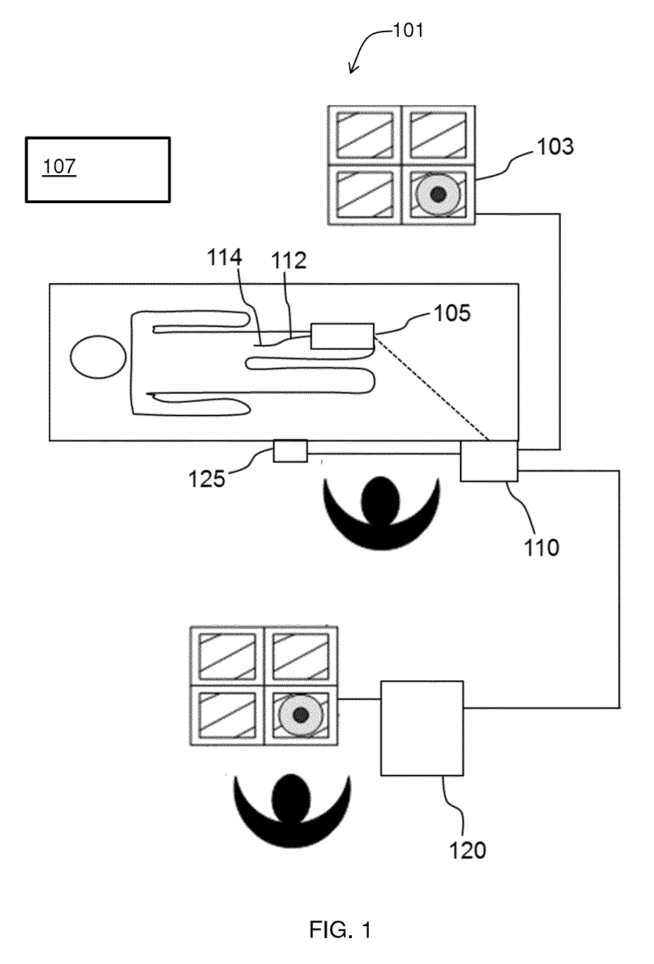

FIG. 1 depicts an exemplary layout of an intravascular imaging system 101 as may be found, for example, in a catheter lab. An operator uses control station 110 and navigational device 125 to operate catheter 112 via patient interface module (PIM) 105. At a distal tip of catheter 112 is imaging tip 114. Computer device 120 works with PIM 105 to coordinate imaging operations. Imaging operations proceed by using catheter 112 to image the patient's tissue. The image data is received by device 120 and interpreted to provide an image on monitor 103. System 101 is operable for use during diagnostic imaging of the peripheral and coronary vasculature of the patient. System 101 can be configured to automatically visualize boundary features, perform spectral analysis of vascular features, provide qualitative or quantitate blood flow data, or a combination thereof.

In some embodiments, operation of system 101 employs a sterile, single use intravascular ultrasound imaging catheter 112. Catheter 112 is inserted into the coronary arteries and vessels of the peripheral vasculature under the guidance of angiogrpahic system 107. System 101 may be integrated into existing and newly installed catheter laboratories (angiography suites.) The system configuration is flexible in order to fit into the existing catheter laboratory work flow and environment. For example, the system can include industry standard input/output interfaces for hardware such as navigation device 125, which can be a bedside mounted joystick. System 101 can include interfaces for one or more of an EKG system, exam room monitor, bedside rail mounted monitor, ceiling mounted exam room monitor, and server room computer hardware.

System 101 connects to catheter 112 via PIM 105, which may contain a type CF (intended for direct cardiac application) defibrillator proof isolation boundary. All other input/output interfaces within the patient environment may utilize both primary and secondary protective earth connections to limit enclosure leakage currents. The primary protective earth connection for controller 125 and control station 110 can be provided through the bedside rail mount. A secondary connection may be via a safety ground wire directly to the bedside protective earth system. Monitor 103 and an EKG interface can utilize the existing protective earth connections of the monitor and EKG system and a secondary protective earth connection from the bedside protective earth bus to the main chassis potential equalization post.

Computer device 120 can include a high performance dual Xeon based system using an operating system such as Windows XP professional. Computer device 120 may be configured to perform real time intravascular ultrasound imaging while simultaneously running a tissue classification algorithm referred to as virtual histology (VH). The application software can include a DICOM3 compliant interface, a work list client interface, interfaces for connection to angiographic systems, or a combination thereof. Computer device 120 may be located in a separate control room, the exam room, or in an equipment room and may be coupled to one or more of a custom control station, a second control station, a joystick controller, a PS2 keyboard with touchpad, a mouse, or any other computer control device.

Computer device 120 may generally include one or more USB or similar interfaces for connecting peripheral equipment. Available USB devices for connection include the custom control stations, the joystick, and a color printer. In some embodiments, control system includes one or more of a USB 2.0 high speed interface, a 50/100/1000 baseT Ethernet network interface, AC power input, PS2 jack, potential equalization post, 1 GigE Ethernet interface, microphone & line inputs, line output VGA Video, DVI video interface, PIM interface, ECG interface, other connections, or a combination thereof. As shown in FIG. 1, computer device 120 is generally linked to control station 110.

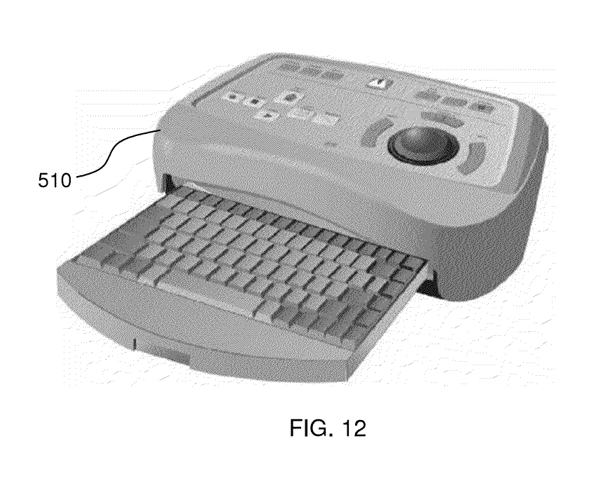

Control station 110 may be provided by any suitable device, such as a computer terminal (e.g., on a kiosk). In some embodiments, control system 110 is a purpose built device with a custom form factor (e.g., as shown in FIG. 12).

In certain embodiments, systems and methods of the invention include processing hardware configured to interact with more than one different three dimensional imaging system so that the tissue imaging devices and methods described here in can be alternatively used with OCT, IVUS, or other hardware.

Any target can be imaged by methods and systems of the invention including, for example, bodily tissue. In certain embodiments, systems and methods of the invention image within a lumen of tissue. Various lumen of biological structures may be imaged including, but not limited to, blood vessels, vasculature of the lymphatic and nervous systems, various structures of the gastrointestinal tract including lumen of the small intestine, large intestine, stomach, esophagus, colon, pancreatic duct, bile duct, hepatic duct, lumen of the reproductive tract including the vas deferens, vagina, uterus and fallopian tubes, structures of the urinary tract including urinary collecting ducts, renal tubules, ureter, and bladder, and structures of the head and neck and pulmonary system including sinuses, parotid, trachea, bronchi, and lungs.

In an exemplary embodiment, the invention provides a system for capturing a three dimensional image by OCT. Commercially available OCT systems are employed in diverse applications such as art conservation and diagnostic medicine, e.g., ophthalmology. OCT is also used in interventional cardiology, for example, to help diagnose coronary artery disease. OCT systems and methods are described in U.S. Pub. 2011/0152771; U.S. Pub. 2010/0220334; U.S. Pub. 2009/0043191; U.S. Pub. 2008/0291463; and U.S. Pub. 2008/0180683, the contents of each of which are hereby incorporated by reference in their entirety.

In OCT, a light source delivers a beam of light to an imaging device to image target tissue. Within the light source is an optical amplifier and a tunable filter that allows a user to select a wavelength of light to be amplified. Wavelengths commonly used in medical applications include near-infrared light, for example between about 800 nm and about 1700 nm.

Generally, there are two types of OCT systems, common beam path systems and differential beam path systems, that differ from each other based upon the optical layout of the systems. A common beam path system sends all produced light through a single optical fiber to generate a reference signal and a sample signal whereas a differential beam path system splits the produced light such that a portion of the light is directed to the sample and the other portion is directed to a reference surface. Common beam path interferometers are further described for example in U.S. Pat. No. 7,999,938; U.S. Pat. No. 7,995,210; and U.S. Pat. No. 7,787,127, the contents of each of which are incorporated by reference herein in its entirety.

In a differential beam path system, amplified light from a light source is input into an interferometer with a portion of light directed to a sample and the other portion directed to a reference surface. A distal end of an optical fiber is interfaced with a catheter for interrogation of the target tissue during a catheterization procedure. The reflected light from the tissue is recombined with the signal from the reference surface forming interference fringes (measured by a photovoltaic detector) allowing precise depth-resolved imaging of the target tissue on a micron scale. Exemplary differential beam path interferometers are Mach-Zehnder interferometers and Michelson interferometers. Differential beam path interferometers are further described for example in U.S. Pat. No. 7,783,337; U.S. Pat. No. 6,134,003; and U.S. Pat. No. 6,421,164, the contents of each of which are incorporated by reference herein in its entirety.

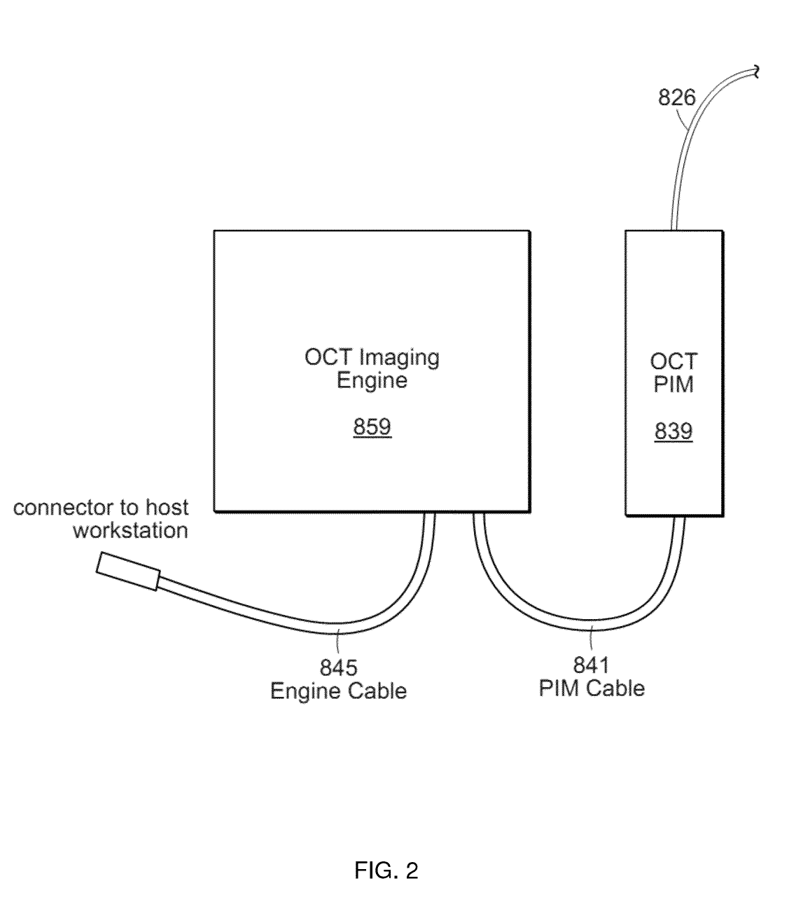

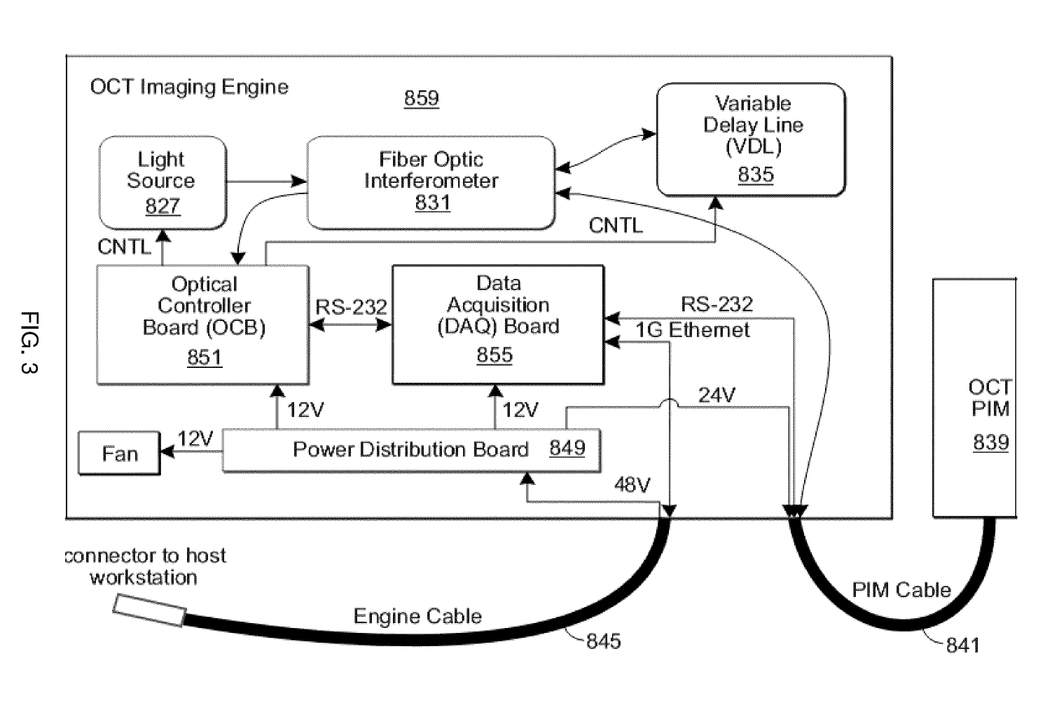

FIG. 2 presents a high-level diagram of a differential beam path OCT system according to certain embodiments of the invention. For intravascular imaging, a light beam is delivered to the vessel lumen via a fiber-optic based imaging catheter 826. The imaging catheter is connected through hardware to software on a host workstation. The hardware includes an imagining engine 859 and a handheld patient interface module (PIM) 839 that includes user controls. The proximal end of the imaging catheter is connected to PIM 839, which is connected to an imaging engine as shown in FIG. 3.

FIG. 3 gives a detailed view of components of imaging engine 859 (e.g., a bedside unit). Imaging engine 859 houses a power supply 849, light source 827, interferometer 931, and variable delay line 835 as well as a data acquisition (DAQ) board 855 and optical controller board (OCB) 854. A PIM cable 841 connects the imagine engine 859 to the PIM 839 and an engine cable 845 connects the imaging engine 859 to the host workstation.

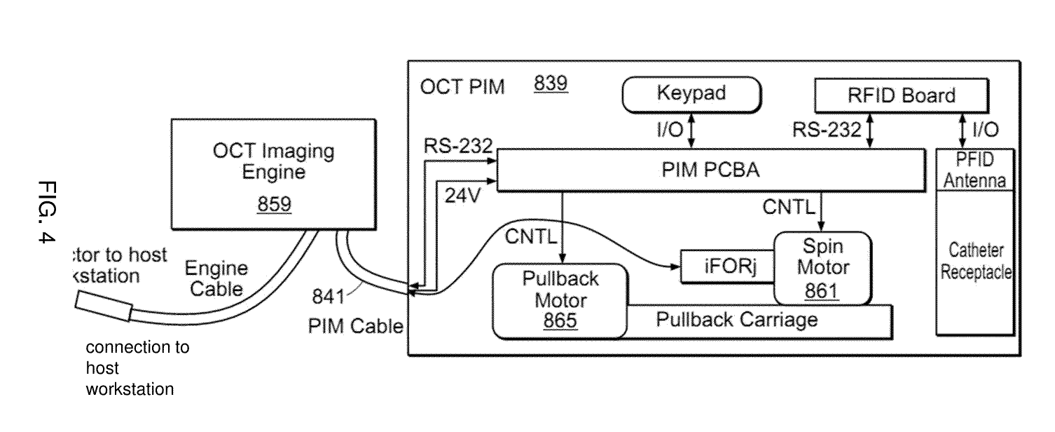

FIG. 4 shows light path in a differential beam path system according to an exemplary embodiment of the invention. Light for image capture originates within the light source 827. This light is split between an OCT interferometer 905 and an auxiliary, or “clock”, interferometer 911. Light directed to the OCT interferometer is further split by splitter 917 and recombined by splitter 919 with an asymmetric split ratio. The majority of the light is guided into the sample path 913 and the remainder into a reference path 915. The sample path includes optical fibers running through the PIM 839 and the imaging catheter 826 and terminating at the distal end of the imaging catheter where the image is captured.

Typical intravascular OCT involves introducing the imaging catheter into a patient's target vessel using standard interventional techniques and tools such as a guide wire, guide catheter, and angiography system. Rotation is driven by spin motor 861 while translation is driven by pullback motor 865.

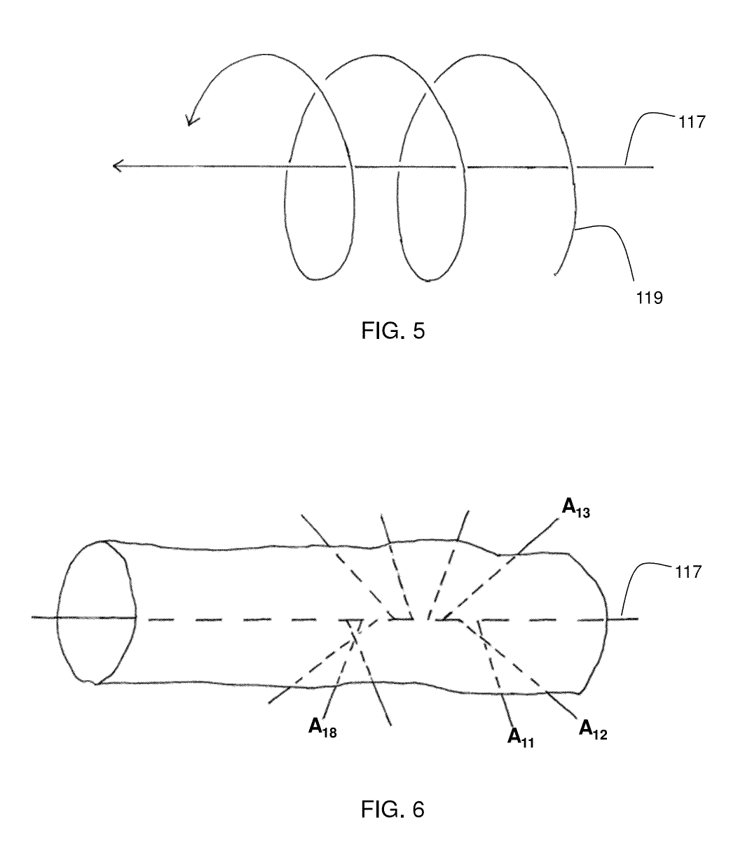

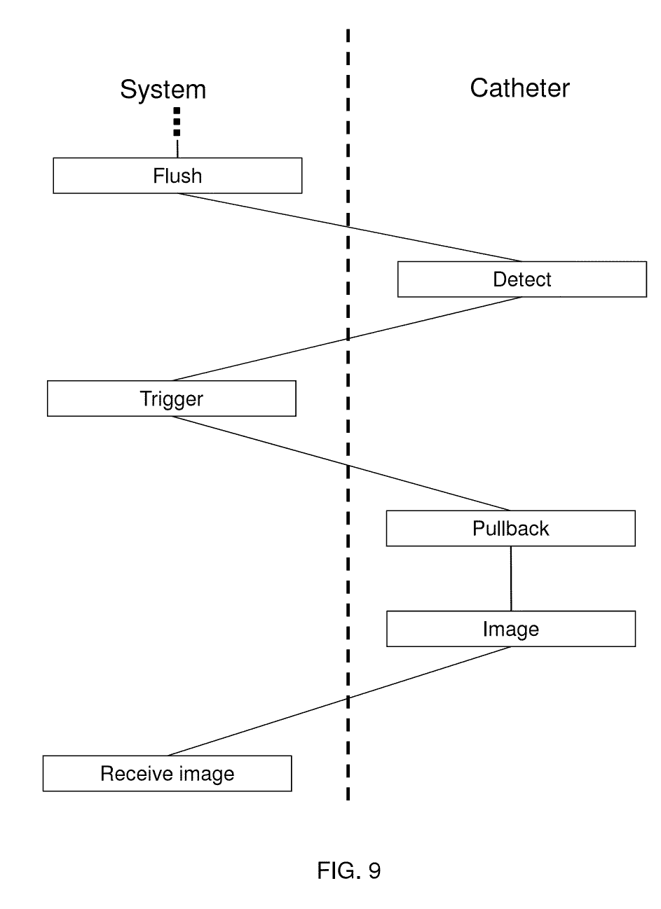

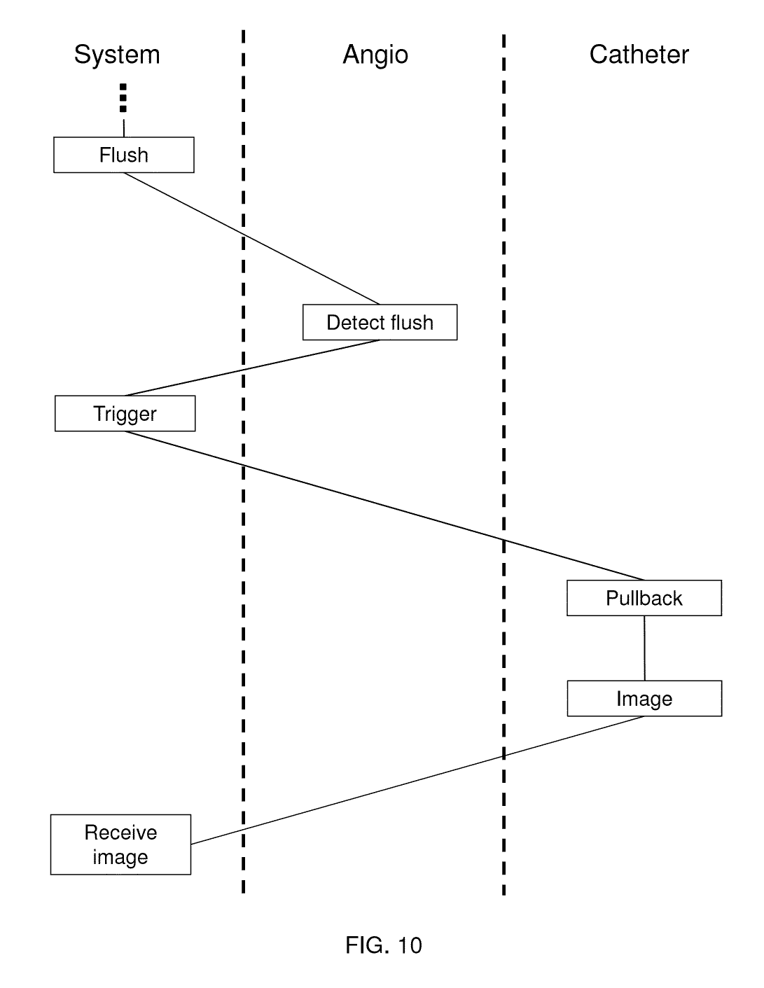

FIG. 5 describes the motion for image capture defined by rotation and translation. Blood in the vessel is temporarily flushed with a clear solution for imaging. Detection of the flushing (see, e.g., FIG. 9 or 10) triggers, via the PIM or control console, the imaging core of the catheter to rotate, pullback, or both while collecting image data that it delivers to the console screen. Using light provided by the imaging engine, the inner core sends light into the tissue in an array of A scan lines as illustrated in FIG. 6 and detects reflected light.

FIG. 6 shows the positioning of A scans with in a vessel. Each place where one of A scans A11, A12, . . . , AN intersects a surface of a feature within vessel 101 (e.g., a vessel wall) coherent light is reflected and detected. Catheter 826 translates along axis 117 being pushed or pulled by pullback motor 865.

The reflected, detected light is transmitted along a sample path of interferometer 831 to be recombined with the light from reference path via a splitter. A variable delay line (VDL) 925 on the reference path uses an adjustable fiber coil to match the length of reference path to the length of sample path. The reference path length may adjusted by a stepper motor translating a minor on a translation stage under the control of firmware or software. The free-space optical beam on the inside of the VDL 925 experiences more delay as the minor moves away from the fixed input/output fiber.

The combined light from the splitter is split into orthogonal polarization states, resulting in RF-band polarization-diverse temporal interference fringe signals. The interference fringe signals are converted to photocurrents using PIN photodiodes on the OCB 851 as shown in FIG. 3. The interfering, polarization splitting, and detection steps are done by a polarization diversity module (PDM) on the OCB. Signal from the OCB is sent to the DAQ 855, shown in FIG. 3. The DAQ includes a digital signal processing (DSP) microprocessor and a field programmable gate array (FPGA) to digitize signals and communicate with the host workstation and the PIM. The FPGA converts raw optical interference signals into meaningful OCT images. The DAQ also compresses data as necessary to reduce image transfer bandwidth to 1 Gbps (e.g., compressing frames with a lossy compression JPEG encoder).

Data is collected from A scans A11, A12, . . . , AN and stored in a tangible, non-transitory memory. A set of A scans generally define a B scan. The data of all the A scan lines together represent a three-dimensional image of the tissue. The data of the A scan lines generally referred to as a B scan can be used to create an image of a cross section of the tissue, sometimes referred to as a tomographic view. The data of the A scan lines is processed according to systems and methods of the inventions to generate images of the tissue. By processing the data appropriately (e.g., by fast Fourier transformation), a two-dimensional image can be prepared from the three dimensional data set. Systems and methods of the invention provide one or more of a tomographic view, ILD, or both.

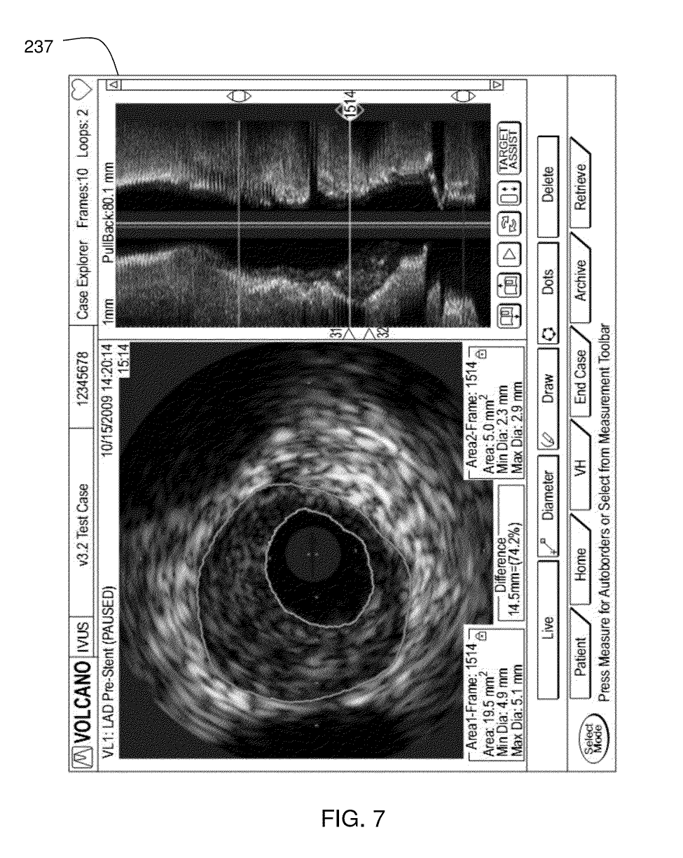

FIG. 7 shows a display 237 including a tomographic view in the left panel. A tomographic view can be represented as a visual depiction of a cross section of a vessel (see left side of FIG. 7). Where a tomographic view generally represents an image as a planar view across a vessel or other tissue (i.e., normal to axis 117), an image can also be represented as a planar view along a vessel (i.e., axis 117 lies in the plane of the view).

FIG. 7 shows a longitudinal planar view of the vessel in the right panel. Such a planar image along a vessel is sometimes referred to as an in-line digital view or image longitudinal display (ILD). The system captures a 3D data set that is used to present the image of tissue. An electronic apparatus within the system (e.g., PC, dedicated hardware, or firmware) stores the three dimensional data set in a tangible, non-transitory memory and renders a display 237 (e.g., on a screen or computer monitor) that includes a 2D image of the tissue.

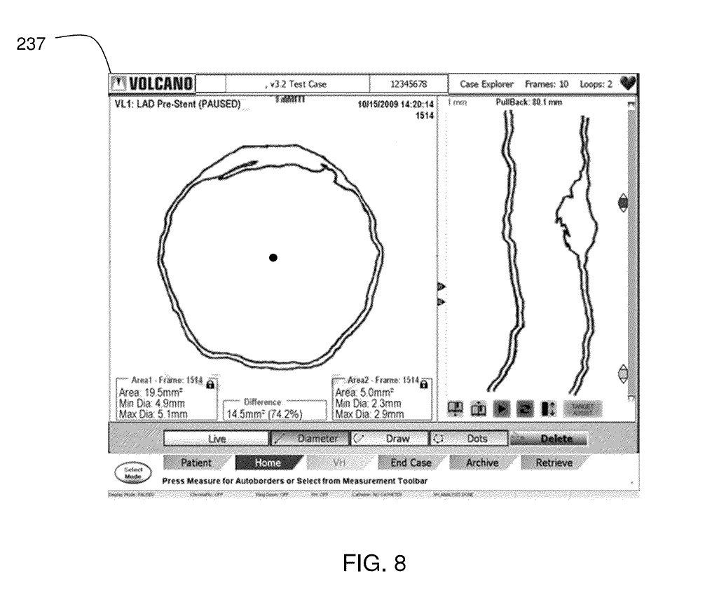

FIG. 8 shows a display similar to that shown in FIG. 7, rendered in a simplified style of the purposes of ease of understanding. Display 237 may be rendered within a windows-based operating system environment, such as Windows, Mac OS, or Linux or within a display or GUI of a specialized system. Display 237 can include any standard controls associated with a display (e.g., within a windowing environment) including minimize and close buttons, scroll bars, menus, and window resizing controls. Elements of display 237 can be provided by an operating system, windows environment, application programing interface (API), web browser, program, or combination thereof (for example, in some embodiments a computer includes an operating system in which an independent program such as a web browser runs and the independent program supplies one or more of an API to render elements of a GUI). Display 237 can further include any controls or information related to viewing images (e.g., zoom, color controls, brightness/contrast) or handling files comprising three-dimensional image data (e.g., open, save, close, select, cut, delete, etc.). Further, display 237 can include controls (e.g., buttons, sliders, tabs, switches) related to operating a three dimensional image capture system (e.g., go, stop, pause, power up, power down). As shown in FIG. 8, display 237 includes two images of tissue, a tomographic view and an ILD. As discussed above, intravascular imaging provides a very good display when blood is flushed from the vessel. The clarity of display 237 as shown in FIG. 7 and drawn in FIG. 8 relates to the ability of the imaging modality to see through the surrounding media and to the affected tissue. In high frequency IVUS, discussed in greater detail below, the imaging involves ultrasonic signals that must penetrate through the media. In OCT, the imaging involves light signals. So that the imaging signal can propagate most directly to the tissue and back, a flush operation replaces the blood with a solution that is transparent to the imaging signal (e.g., saline). Thus, the medium surrounding the image capture device does not interfere with the imaging operation. Additionally, so that image capture is well synchronized and a useful data set can be captured on every operation, the flush operation is used as the direct trigger of the image capture operation.

FIG. 9 diagrams a method of flush-triggered imaging applicable in, for example, OCT. Once the imaging catheter is inserted to the site to be imaged, a detection mechanism on or near the imaging catheter is used. The detection mechanism can be the light path of the OCT system itself, a dedicated pressure sensor on the OCT catheter, an angiographic system, or any other suitable system. Since successfully flushing blood out of a vessel can involve pushing in a solution at a higher pressure than the blood, a pressure detector can be used to detect the flush. Intravascular blood pressure detectors can operate via piezoelectric or similar detection elements. Suitable blood pressure detectors are discussed in U.S. Pat. No. 7,335,161; U.S. Pat. No. 6,886,411; U.S. Pat. No. 6,504,286; U.S. Pat. No. 5,873,835; U.S. Pub. 2009/0270695; and U.S. Pub. 2005/0197585, the contents of which are incorporated by reference herein in their entirety. The influx of solution causes a clearly-detectable spike in blood pressure. In the vessel, at or near the catheter, the change in blood pressure is detected. The pressure detector need not be directly on the catheter (although that may be one suitable place for it). It may be on a dedicated catheter, on a guidewire, implanted, injected, or otherwise positioned.

Where the detection mechanism is optical—for example, the OCT light path and detection circuitry is used to detect the displacement of blood by solution (e.g., transition from dark to light), the OCT imaging tip is operating optically as the solution is flushed in. A processor in the OCT imaging engine can detect a change in light by digital signal processing techniques. Whether the detection is optical, pressure based, ultrasound based, other, or a combination thereof, the detection at the catheter end of the system operates as a trigger at the control end of the system to initiate the OCT catheter pullback. During pullback, the OCT systems captures an image of the tissue (e.g., the in the form of a 3D data set) by sending the interferometric signal back to the system. The system receives the image and processes it for storage or presentation as a display 237. Additionally or alternatively, the flush can be detected from outside of the vessel (e.g., outside of the body). Any suitable external detection method can be employed, such as a blood pressure cuff or an angiography system.

FIG. 10 shows a method for angio-triggered imaging. Here, an angiography system is used with the imaging system (e.g., OCT, IVUS, or optical-acoustic imaging). Angiography systems can be used to visualize the blood vessels by injecting a radio-opaque contrast agent into the blood vessel and imaging using X-ray based techniques such as fluoroscopy. Angiographic techniques include projection radiography as well as imaging techniques such as CT angiography and MR angiography. In certain embodiments, angiography involves using a catheter to administer the x-ray contrast agent at the desired area to be visualized. The catheter is threaded into an artery, and the tip is advanced through the arterial system into the major coronary artery. X-ray images of the transient radio contrast distribution within the blood flowing within the coronary arteries allows visualization of the size of the artery openings. Features and media within the blood and walls of the arteries are studied. Angiography systems and methods are discussed, for example, in U.S. Pat. No. 7,734,009; U.S. Pat. No. 7,564,949; U.S. Pat. No. 6,520,677; U.S. Pat. No. 5,848,121; U.S. Pat. No. 5,346,689; U.S. Pat. No. 5,266,302; U.S. Pat. No. 4,432,370; and U.S. Pub. 2011/0301684, the contents of each of which are incorporated by reference in their entirety for all purposes.

As shown in FIG. 10, the angiography system can be used to detect a change. The angiography system can be used to detect the flush with saline (e.g., the temporary displacement of the radiopaque dye by the saline), the initial influx of radiopaque dye, or other such flushes. A processor that receives the angiography signal data can detect a brightness or contrast change (e.g., by digital signal processing techniques including those described in Smith, 1997, THE SCIENTIST AND ENGINEERS GUIDE TO DIGITAL SIGNAL PROCESSING, California Technical Publishing (San Diego, Calif.) 626 pages, the contents of which are hereby incorporated by reference). The imaging system uses the flush detection as a trigger to initiate pullback and image capture via the imaging catheter. The imaging system then receives the image.

Flush triggered imaging may have particular application in IVUS. For example, high-frequency IVUS can detect speckling from the blood and can benefit from flushing the blood from the system with a clear (to IVUS) solution. In certain embodiments, the invention provides systems and methods for flush-triggered IVUS imaging.

IVUS uses a catheter with an ultrasound probe attached at the distal end. The proximal end of the catheter is attached to computerized ultrasound equipment. To visualize a vessel via IVUS, angiographic techniques are used and the physician positions the tip of a guide wire, usually 0.36 mm (0.014″) diameter and about 200 cm long. The physician steers the guide wire from outside the body, through angiography catheters and into the blood vessel branch to be imaged.

The ultrasound catheter tip is slid in over the guide wire and positioned, again, using angiography techniques, so that the tip is at the farthest away position to be imaged. Sound waves are emitted from the catheter tip (e.g., in about a 20-40 MHz range) and the catheter also receives and conducts the return echo information out to the external computerized ultrasound equipment, which constructs and displays a real time ultrasound image of a thin section of the blood vessel currently surrounding the catheter tip, usually displayed at 30 frames/second image.

The guide wire is kept stationary and the ultrasound catheter tip is slid backwards, usually under motorized control at a pullback speed of 0.5 mm/s. Systems for IVUS are discussed in U.S. Pat. No. 5,771,895; U.S. Pub. 2009/0284332; U.S. Pub. 2009/0195514 A1; U.S. Pub. 2007/0232933; and U.S. Pub. 2005/0249391, the contents of each of which are hereby incorporated by reference in their entirety. Imaging tissue by IVUS produces tomographic (cross-sectional) or ILD images, for example, as illustrated in FIG. 8 and shown in FIG. 7. An IVUS system can be installed substantially as shown in FIG. 1. An IVUS computer device 520 takes the place of OCT computer device 120.

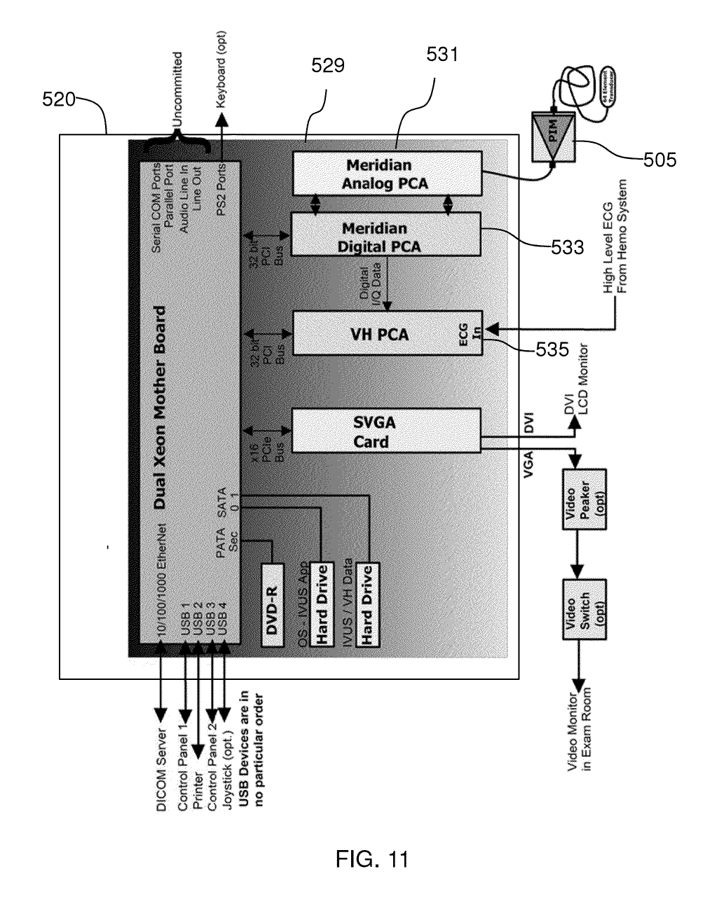

FIG. 11 describes an exemplary computer device 520 according to certain embodiments. Computer device 520 may include a motherboard 529 that includes an IVUS signal generation and processing system. The signal generation and processing system may comprises an analog printed circuit assembly (PCA) 131, an digital PCA 133, one or more filter modules, and a VH board 535. Analog PCA 131 and digital PCA 133 are used to excite transducer 514 via catheter 512 and to receive and process the gray scale IVUS signals. The VH board 535 is used to capture and pre-process the IVUS RF signals and transfer them to the main VH processing algorithm as run by a computer processor system (e.g., dual Xeon processors). PIM 105 is directly connected to the analog PCA 131.

FIG. 12 shows a control station 510 according to certain embodiments. A slide out keyboard is located on the bottom for manual text entry. Control station 510 may be designed for different installations options. The station can be placed directly on a desktop surface. With an optional bedside mounting kit, control station 510 can be affixed directly to the bedside rail. Control station 510 can include a standard four hole VESA mount on the underside to allow other mounting configurations. Control system 510 may provide a simple-to-use interface with frequently-operated functions mapped to unique switches. Control station 510 may be powered from, and may communicate with, computer 520 using a standard USB 1.1 interface. The system may include a control panel 515. In some embodiments, multiple control panels 515 are mounted in both the exam room and/or the control room. Control system 510 can have a surface control panel with buttons for frequently-operated functions (e.g., as contact closure switches). Those dome switches are covered with a membrane overlay. The use of dome switches provides a tactile feedback to the operator upon closure. The control panel may include a pointing device such as a trackball to navigate a pointer on the graphical user interface of the system. The control panel may include several screen selection keys. The settings key is used to change system settings like date and time and also permits setting and editing default configurations. The display key may be used to provide enlarged view for printing. In some embodiments, the print key prints a 6×4 inch photo of the current image on the screen. The control panel may include a Ring Down key that toggles the operation of ringdown subtraction. A chroma key can turn blood flow operations on and off. The VH key can operate the virtual histology engine. A record, stop, play, and save frame key are included for video operation. Typically, the home key will operate to display the live image. A menu key provides access to measurement options such as diameter, length, and borders. Bookmark can be used while recording a loop to select specific areas of interest. Select (+) and Menu (−) keys are used to make selections.

In some embodiments, the system includes a joystick for navigational device 525. The joystick may be a sealed off-the-shelf USB pointing device used to move the cursor on the graphical user interface from the bedside. System 501 may include a control room monitor, e.g., an off-the-shelf 59″ flat panel monitor with a native pixel resolution of 5280×1024 to accept DVI-D, DVI-I and VGA video inputs.

Control station 510 is operably coupled to PIM 105, from which catheter 512 extends. Catheter 512 includes an ultrasound transducer 514 located at the tip. Any suitable IVUS transducer may be used. For example, in some embodiments, transducer 514 is driven as a synthetic aperture imaging element. Imaging transducer 514 may be approximately 5 mm in diameter and 2.5 mm in length. In certain embodiments, transducer 514 includes a piezoelectric component such as, for example, lead zirconium nitrate or PZT ceramic. The transducer may be provided as an array of elements (e.g., 64), for example, bonded to a Kapton flexible circuit board providing one or more integrated circuits. This printed circuit assembly may rolled around a central metal tube, back filled with an acoustic backing material and bonded to the tip of catheter 514. In some embodiments, signals are passed to the system via a plurality of wires (e.g., 7) that run the full length of catheter 512. The wires are bonded to the transducer flex circuit at one end and to a mating connector in PIM 105 at the other. The PIM connector may also contains a configuration EPROM. The EPROM may contain the catheter's model and serial numbers and the calibration coefficients which are used by the system. The PIM 105 provides the patient electrical isolation, the beam steering, and the RF amplification. PIM 105 may additionally include a local microcontroller to monitor the performance of the system and reset the PIM to a known safe state in the event of loss of communication or system failure. PIM 105 may communicate with computer device 520 via a low speed RS232 serial link.

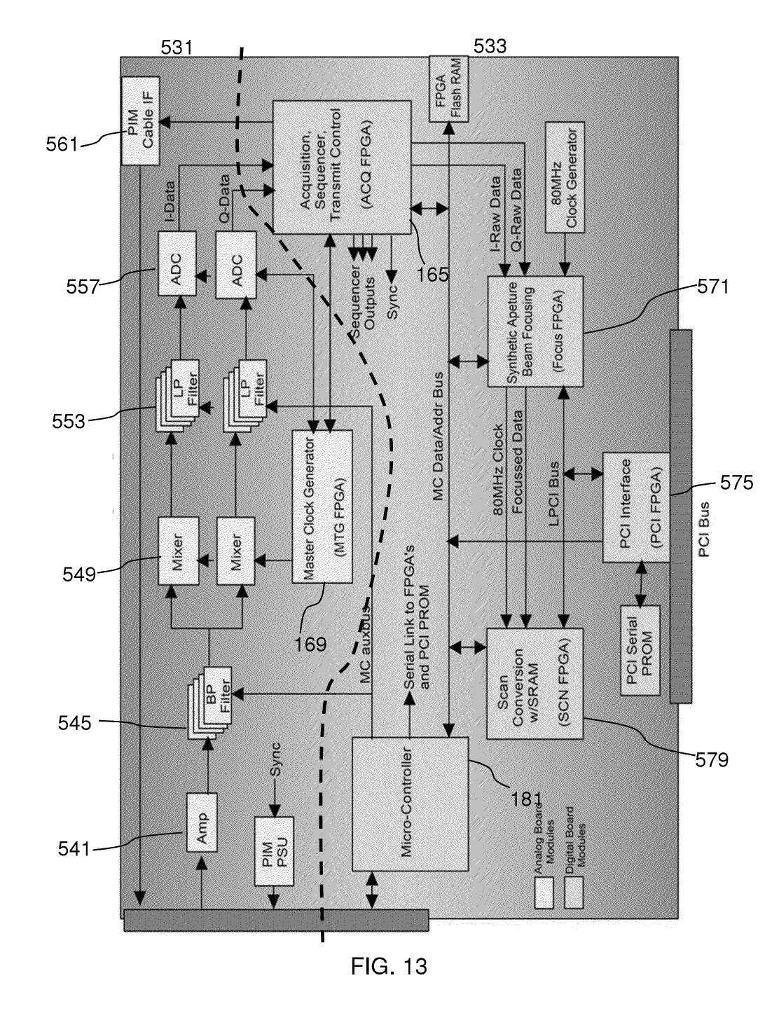

FIG. 13 provides a schematic of analog PCA 131 and digital PCA 133 according to certain embodiments of the invention. Analog PCA 131 is shown to include amplifier 541, band pass filter 545, mixer 549, low pass filter 553, and analog-to-digital converter (ADC) 157. (Here, the system is depicted as being operable to convert the transducer RF data to “In-Phase” and “Quadrature” (IQ) data. According to this embodiment, ADC 157 is 52-bits wide and converts the IQ data to a dual digital data stream.) Analog board 531 further includes an interface module 561 for PIM 105, as well as a clock device 569.

Digital PCA 133 is depicted as having an acquisition FPGA 165, as well as a focus FPGA 171, and a scan conversion FPGA 179. Focus FPGA 171 provides the synthetic aperture signal processing and scan conversion FPGA 179 provides the final scan conversion of the transducer vector data to Cartesian coordinates suitable for display via a standard computer graphics card on monitor 503. Digital board 533 further optionally includes a safety microcontroller 581, operable to shut down PIM 105 as a failsafe mechanism. Preferably, digital PCA 133 further includes a PCI interface chip 575. It will be appreciated that this provides but one exemplary illustrative embodiment and that one or skill in the art will recognize that variant and alternative arrangements may perform the functions described herein. Clock device 569 and acquisition FPGA 165 operate in synchronization to control the transmission of acquisition sequences. FIG. 13 presents one exemplary system architecture, and other IVUS systems are known in the art and may be used for flush-triggered IVUS imaging. For flush-triggered imaging, the blood is displaced by a solution, and this flushing step is detected. Any suitable detection mechanism can be used including, for example, blood pressure or angio systems as discussed above.

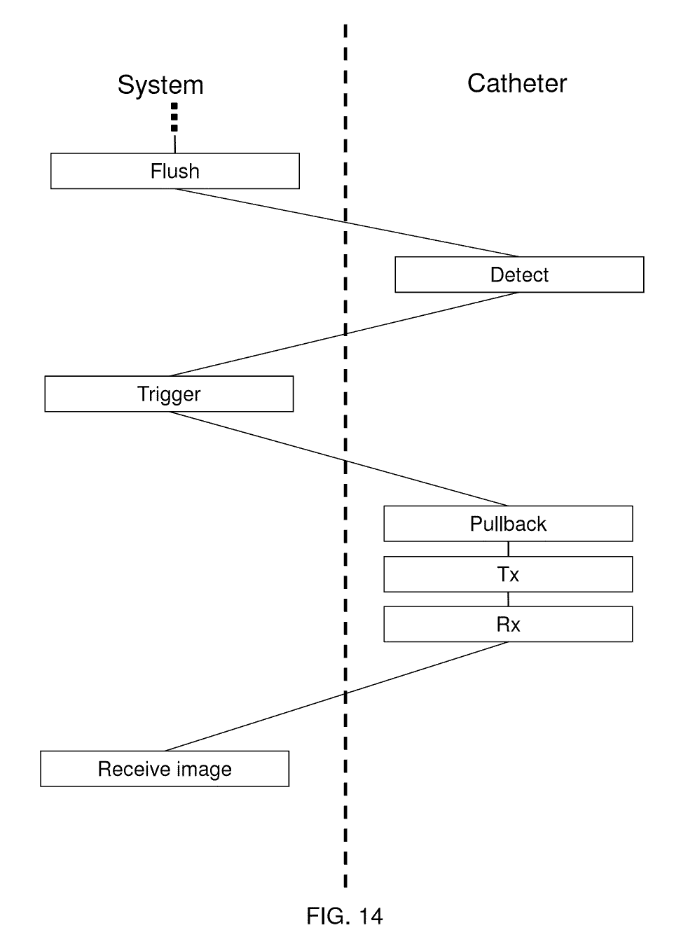

FIG. 14 is a diagram of a flush-triggered IVUS imaging method. The imaging catheter is inserted and the system is readied. Blood is flushed out of the relevant portion of the vessel to allow for the clearest image capture (i.e., this can provide a de-speckling step, or speckle prevention, for high frequency IVUS). Flushing may have particular benefit in high-frequency IVUS by allowing the luminal border to be detected. Much of the field of intravascular imaging is concerned with the lumen of a vessel. The stenosis, or narrowing, of arteries (i.e., decrease in luminal border) is a very significant health problem associated with atherosclerosis, vulnerable plaque, strokes, and heart attacks. The ability to accurately see the luminal border can provide a life-saving diagnostic tool and flushing the blood to prevent blood speckle can provide the ability to see the luminal border if the pullback operation is properly coordinated with the flushing. Accordingly, as diagramed in FIG. 14, the flush can be detected by a device at or near the catheter, such as a blood pressure sensor on the catheter itself or by an ultrasound transmit (Tx) and receive (Rx) pulse. Detection of the flush is used by the system as a trigger to initiate pullback of the catheter. During pullback, one or a series of Tx and Rx pulses can be performed to capture an image (e.g., in the form of a series of A lines). The system receives the image data set and performs any desired processing and can then provide the image for medical diagnostics.

Because the imaging is triggered by the flush, the pullback operation is properly coordinated with the removal of the blood. Additionally or alternatively, the flush detection may be performed through the use of an angiographic system.

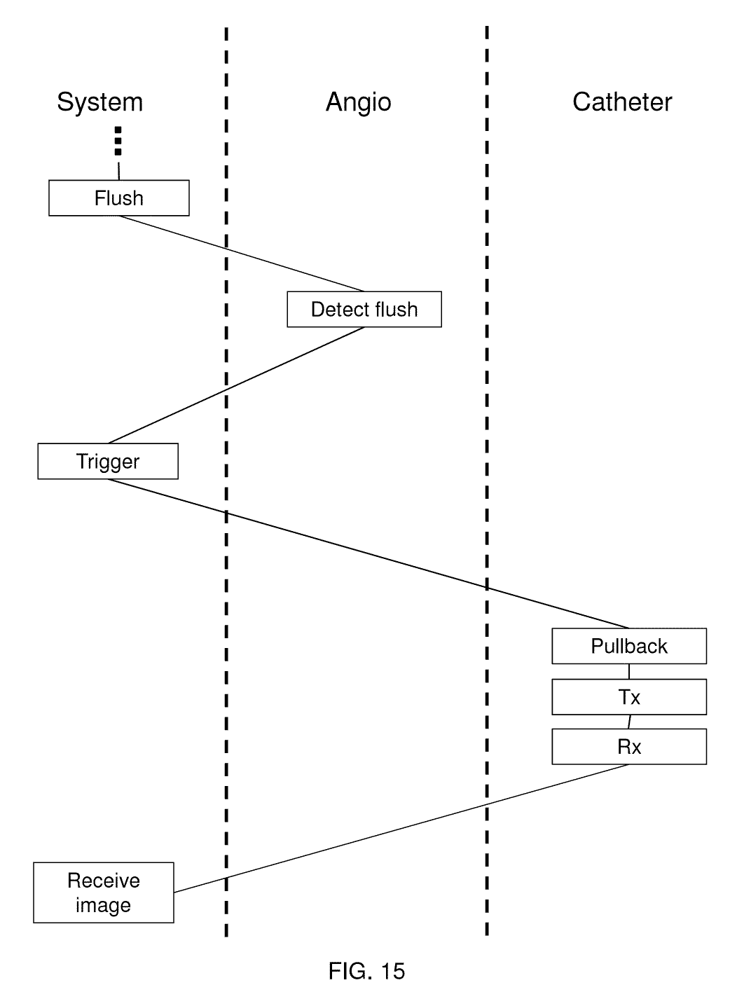

FIG. 15 illustrates angio-triggered IVUS imaging. The use of angiographic systems is discussed above. Once the system initiates the flush, the angio system is used to detect the flush. The system uses the angio detection as a trigger to initiate a catheter pullback. The pullback is associated with Tx and Rx operations that capture an image. The image is received by the system for medical diagnostics. IVUS systems are discussed in U.S. Pub. 2012/0271175; U.S. Pub. 2012/0271170; U.S. Pub. 2011/0160586; U.S. Pub. 2009/0284332; U.S. Pub. 2009/0043191; U.S. Pub. 2008/0281205; and U.S. Pub. 2001/0007940, the contents of which are incorporated by reference in their entirety for all purposes.

While discussed above in terms of flushing blood with a solution that can be clear, flush triggering is applicable to any flushing of a vessel. In some embodiments, the invention provides the coordination of angiography with intravascular imaging by using the injection of a radiopaque dye (e.g., for angiography) as the trigger for the intravascular imaging operation. It will be appreciated that methods described herein can be used to coordinate angiography to intravascular imaging. The angio system can detect the influx of radiopaque dye and use that detection as the trigger for an imaging operation. Additional discussion can be found in U.S. Pat. No. 8,208,995 to Tearney; U.S. Pat. No. 6,947,787 to Webler; U.S. Pat. No. 6,134,003 to Tearney; U.S. Pat. No. 4,998,972 to Chin; and U.S. Pub. 2011/0077528 to Kemp, the contents of each of which are incorporated by reference.

Other embodiments are within the scope and spirit of the invention. For example, due to the nature of software, functions described above can be implemented using software, hardware, firmware, hardwiring, or combinations of any of these. Features implementing functions can also be physically located at various positions, including being distributed such that portions of functions are implemented at different physical locations.

Steps of the invention may be performed using dedicated medical imaging hardware, general purpose computers, or both. As one skilled in the art would recognize as necessary or best-suited for performance of the methods of the invention, computer systems or machines of the invention include one or more processors (e.g., a central processing unit (CPU) a graphics processing unit (GPU) or both), a main memory and a static memory, which communicate with each other via a bus. A computer device generally includes memory coupled to a processor and operable via an input/output device.

Exemplary input/output devices include a video display unit (e.g., a liquid crystal display (LCD) or a cathode ray tube (CRT)). Computer systems or machines according to the invention can also include an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Memory according to the invention can include a machine-readable medium on which is stored one or more sets of instructions (e.g., software), data, or both embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any of the methodologies of the present invention. The term “machine-readable medium” shall accordingly be taken to include, but not be limited to, solid-state memories (e.g., subscriber identity module (SIM) card, secure digital card (SD card), micro SD card, or solid-state drive (SSD)), optical and magnetic media, and any other tangible storage media. Preferably, computer memory is a tangible, non-transitory medium, such as any of the foregoing, and may be operably coupled to a processor by a bus. Methods of the invention include writing data to memory—i.e., physically transforming arrangements of particles in computer memory so that the transformed tangible medium represents the tangible physical objects—e.g., the arterial plaque in a patient's vessel.

As used herein, the word “or” means “and or or”, sometimes seen or referred to as “and/or”, unless indicated otherwise.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of intravascular imaging, the method comprising:
positioning a distal end of an imaging catheter within a blood vessel and immersed in blood, wherein a proximal end of the catheter is connected to an imaging system;
providing an influx of a solution to flush the blood from the vessel;
detecting the influx; and
using the detection of the influx of the solution to trigger an imaging operation with the imaging catheter.

2. The method of claim 1, wherein the influx is detected from within the vessel.

3. The method of claim 1, wherein the influx is detected within the vessel from outside of the patient's body.

4. The method of claim 1, wherein the imaging system is an OCT system.

5. The method of claim 1, wherein the imaging system is an IVUS system.

6. The method of claim 1, wherein detecting the influx comprises detecting an optical change via OCT.

7. The method of claim 1, wherein detecting the influx comprises detecting a pressure change via a pressure sensor on an IVUS catheter.

8. The method of claim 1, wherein detecting the influx comprises detecting a change on an angiogram.

9. The method of claim 1, wherein the imaging operation comprises a pullback.

10. The method of claim 1, wherein the imaging system is configured to initiate a catheter pullback automatically and in direct response to the detection of the influx.

11. A system for intravascular imaging, the system comprising:
an imaging instrument;
a catheter comprising a proximal end connected to the imaging instrument and a distal end configured for insertion into a blood vessel of a patient;
a lumen extending along the catheter for delivery of a solution to flush blood from the blood vessel; and
a sensor to detect an influx of the solution, wherein the system is configured to use the detection of the influx of the solution to trigger an imaging operation with the imaging catheter.

12. The system of claim 11, wherein the influx is detected from within the vessel.

13. The system of claim 11, wherein the influx is detected within the vessel from outside of the patient's body.

14. The system of claim 11, wherein the imaging instrument comprises an OCT device.

15. The system of claim 11, wherein the imaging instrument comprises an IVUS device.

16. The system of claim 11, wherein detecting the influx comprises detecting an optical change via OCT.

17. The system of claim 11, wherein the catheter further comprises a pressure sensor operable to detect the influx.

18. The system of claim 11, further comprising an angiography device operable to detect the influx.

19. The system of claim 11, wherein the imaging operation comprises a pullback.

20. The system of claim 11, wherein the imaging instrument is configured to initiate a catheter pullback automatically and in direct response to the detection of the influx.
